(19) United States
(12) Patent Application Publication
(10) Pub. No.: US 2015/0151042 A1
(43) Pub. Date: Jun. 4, 2015

(54) PROSTHESIS HAVING CONTROL FUNCTION

(71) Applicant: MAXEN MEDICAL CO., LTD., Gwangju (KR)

(72) Inventor: Kyung Hun Han, Seongnam (KR)

(21) Appl. No.: 14/548,299

(22) Filed: Nov. 20, 2014

(30) Foreign Application Priority Data
Dec. 2, 2013 (KR) 10-2013-0148671
May 15, 2014 (KR) 10-2014-0058230

(51) Int. Cl. A61M 5/142; A61F 2/02; A61M 5/158

(52) U.S. Cl. CPC A61M 5/1428; A61M 5/158; A61F 2/02; A61M 2210/167

(57) ABSTRACT

The present invention relates to a prosthesis having a control function, which can be implanted under the skin of a human body and an animal and can be controlled so as to intentionally and intermittently secrete drugs stored in a chamber and a lumen. The prosthesis has an exterior of an I-shape or a C-shape when viewed from top. The prostheses can be used for drug administration and treatment in male erectile dysfunction, treatment of hypersensitivity reaction for penis dwarfism and dwarfism, drug administration in conversion from off-state to on-state for the treatment of Parkinson's syndrome, drug administration in treatment necessary for multi-frequency and long-time administration, administration of drugs reduced or lost in pharmacological activity in digestive system, drug administration after stereotactic surgery for brain disease, and drug administration in a treatment method of directly administering drugs on a lesion to avoid delayed effect of drug.

### CROSS REFERENCE TO PRIOR APPLICATIONS

This application claims priority of Korean Patent Application No. 10-2013-0148671 filed on Dec. 2, 2013 and Korean Patent Application No. 10-2014-0058230 filed on May 15, 2014, which are all hereby incorporated by reference in their entirety.

### TECHNICAL FIELD

The following disclosure relates to a prosthesis inserted or implanted under the skin of a human body or an animal, and in particular, to a prosthesis having a control function, which is implanted into a hypodermic layer and allows a drug stored in the prosthesis to be intentionally and intermittently secreted according to a will of a user.

### BACKGROUND ART

Primary attempts to treat impotence are an oral medicine administration, topical application of drug and insertion of drug into urinary track. Additionally, there are a treatment to put a vacuum devise on the outside of a penis and psychological treatment. Secondary attempts include injection of a vasoactive material in cavernous body of penis and blood vessel reconstruction surgery. Even more active attempts are to insert or implant a machine equipped with a pump and a vacuum cylinder in and around a penis, and also to insert or implant a flexible or expandable prosthesis in a penis.

The most easily accessible treatment method for impotent patients is an oral administration method. Available oral administration agents include phentolamine (e.g., from Vasomax™ and Zonagen) and a variety of phosphodiesterse type5 inhibitors.

The most noninvasive and easiest treatment method for impotence is an oral medicine administration method, but this oral medicine administration method has —shortcoming of “first pass effect”. When the drug is taken through the mouth, the drug passes through intestinal cells, liver portal vein, and liver, and ends up circulating in the body a few times. The drug then undergoes moving and deactivation process a few times and only a fraction of the drug reaches the target spot for application by systematic circulation. For this reason, much less amount of the drug than dosage reaches application spot that serves the function. Oral medicine administration has a shortcoming that the agent imposes undesirable load onto digestive organs such as the stomach, intestine, liver and cholecyst.

In addition to first pass effect of oral agent use, the agent may stimulate the gastro-intestinal tract with its activated or non-activated components and may have a harmful effect on the body of the user. Oral agent use has a disadvantage against the agent's topical application in that the use may have partially harmful effect on the body before the drug reaches a target spot to cure. For example, Vasomax™ which induces erection by use of blocking effect of adrenergic receptors is a contraindicative drug for patients with unstable nervous system and cardiovascular system. Oral agent use for disorders in general as well as specifically for sexual function disorders needs improvement in medication method or tool which can help avoid the first pass effect and disadvantages of bodily harm.

In addition to oral administration method for the impotence treatment, there are other administration methods through the penis skin or mucous membrane of urethra. These methods include drug topical application through epidermis of glans penis, penile shaft, scrotum, fossa navicularis of glans penis and urethra membrane. Manufacturing methods of the semi-solid drug to be inserted through fossa navicularis or urethra membrane and the method of its medication are disclosed in International application publication WO 03/070281 A1. A drawback to this treatment is the partner of a sexual intercourse may suffer bad effect due to possible contact with the drug during the intercourse. Lag time for the drug to take effect is a major drawback of this treatment. In addition, an insufficient amount of the drug may well be administered for inducing and maintaining erection. There are fewer side effects with the treatment than cavernous body injection. However, 10 times as much prostaglandin E1 is needed for the same effect. It takes the drug considerable amount of time to penetrate stratum corneum or membrane and be absorbed into the body, thus delaying the drug from taking effect.

It is reported in literature that only 20% to 30% of patients whom received the urethra medication to responded to the treatment and 70% eventually give up the treatment. The semi-solid drug is inserted into urethra by a depth of 3 cm. Pain and wound due to this insertion is another drawback of this treatment. A medication system is necessary which may solve the phenomenon of delayed effect of medication through the skin or the membrane.

When the drawback of oral agent use turns out outstanding during impotence treatment, drug injection into corpus cavernosum may be considered. Such way of medication to administer straight to the target has an advantage to minimize the amount of the drug, unlike oral intake or percutaneous infiltration. Also, a research paper on erection results of prostaglandin E1 injection into penile corpus cavernosum of spinal cord injury patients and another research paper on sexual dysfunction disorders of spinal cord injury patients read that penile corpus cavernosum injection or penile prosthesis implantation helps spinal cord injury patients to recover their sexual function.

However, penile corpus cavernosum injection, though having many merits, has its own constraints and limitations. Academic literature published by Althof et al. was a report about the process and finding of a research on penile corpus cavernosum injection of a medicine containing papaverine and phentolamine. 84% of the research object patients showed improved erection, but 57% of them gave up their support for the research. 25% of the object patients for the research have fibrosis of their penile corpus cavernosums or small fibrous tumor growths in them, 30% of the object patients show undesirable liver somatic index increase, and 19% of them have the injected part wounded and infected. According to another report, rate of patients who give up the treatment, corpus cavernosum injection, within 6 months was 30% to 40%. Major side effects of corpus cavernosum injection include unwanted sustained erection, cavernous body turning fibrous or fibrous tumors outbreak in the body, deflected distortion of penis, plaque or hard lump, systemic infection, parenchyma necrosis, pain and panic, and momentary hypotension. Corpus cavernosum injection increases the probability that the patient and his sexual intercourse partner are exposed to contagious disease because the intercourse occurs with his skin barrier torn. Viral diseases including AIDS may have fatally bad effect on the bodies and bacterial infection which is much more possible does harm to the whole body system. There arises a need of medication method which may improve the limitations of corpus cavernosum injection.

One of commercially available and also expectant invasive treatments is a treatment where a machine equipped with a cylinder and a vacuum pump is inserted and implanted into and around a penis. A treatment method to insert an extendible and expandable cylinder into penile corpus cavernosum or under the skin of penis and a device used for the treatment are disclosed in many patents. International application publication WO 01/47441 A2 and EU Patent No. 0137752 are patents to seek expansion and extension by implanting prosthesis in a penis. However, these patents do not have functions to store and secrete drug. The treatment which implants a machine or an apparatus has a few disadvantages together with many advantages. There is a disadvantage in that the patient needs to have a serious surgery and fears from the operation, pain before and after the implantation, infection and remaining scar. There may occur economic losses due to the expenses for repair or removal for malfunctioning and costly price of the equipment. Other disadvantages include sense of being interrupted during the intercourse by the attached machine, short validity period of the implanted machine and deformity damages on tissues in case of long-term use.

As has been previously noted before, when oral agent use is restricted to impotence patients including spinal cord injury patients, when medication administered through the skin or the membrane does not give satisfactory results, when repetitive corpus cavernosum injections or mechanical implantation of prosthesis are not the best solution, there is a need yet for a new treatment different from the typical treatments and also for improvements to be presented about the treatments.

Prostaglandin E1 is an important drug stored in and secreted from the prosthesis which is an embodiment of the present invention and required by the invention to have fluidity property. Prostaglandin E1 is a poorly water-soluble material and a chemically very unstable drug. The chemical instability put limitation to the effort to store Prostaglandin E1 at room temperature and in a liquid state. Korean Patent No. 1003501790000 is a patent granted to GUJU Pharma. Co. Ltd., Seoul, about a manufacturing method of Prostaglandin E1 drug. This patent is about a composite of Prostaglandin E1 whose chemical stability is largely improved in a liquefied drug state. Manufacturing method of Prostaglandin E1-contained drug which has 32.5 days of shelf life (t90%) at 40 degrees in Celcius, i.e., higher than body temperature is disclosed in the patent. The drug stored in and secreted from the prosthesis of this embodiment has to satisfy the conditions of heat resistance at temperatures over body temperature, extension of preservation period, liquid property and no cross interaction among drugs in addition to the basic requirement that it is good for a disease cure.

Among the therapies which require repetitive long-term injections is growth hormone injection against dwarfism. Oral medication is being partially attempted, but hypodermic injection is common. Due to the first pass effect barrier and administered drug's chemical instability, growth hormone administration is repeated daily for a period of at least 6 months to 3 years by injection. Limitations of a skin deformation and pain around the injection area, an increasing fear during childhood, a burden of repetitively ongoing hospital visits, and an increase of hospital visits and treatment cost occur as a result. Therapies requiring repetitive injections need a new method of administration devise.

Regarding oral administration of medication for terminal stage patient of Parkinson's disease, an increasing frequency of daily medication due to the acquisition of drug resistance is an annoying limitation. It even happens that the number of times to administer medication of anti-Parkinsonism drug including dopamine and dopamine agonist increase to over 6 times per day as struggle with the disease is prolonged. Polymer device containing dopamine and/or dopamine agonist with the objectives of improvement of reduced compliance and reduction of medication frequency of anti-Pakinsonism drug is being disclosed in International application publication WO 2004/089375 A1. In the patent a method to manufacture the polymer which allows dopamine and/or dopamine agonist to be secreted through small holes on capsulized matrix is disclosed. The patent claims that there should be a method to cope with the limitation of increasing medication frequency to terminally ill Parkinson's disease patients and discloses hypodermic medication by use of polymer device as the method. The method of the hypodermic medication by use of polymer matrix of this patent is a passive method, which does not have the function to regulate the drug secretion in a purposeful and intermittent way. The method does not have the function to resupply the drug by way of puncturing skin, either. There is need for yet another treatment tool which can improve the limitations arising from frequent medications to Parkinson's disease patients.

During oral medication, Parkinson's disease patients show two opposite cross-reactions of “Off” state where the patient has hypokinesia or dyskinesia and “On” state where the patient shows hypermobility or muscle relaxation. Parkinson's disease patients who reach the terminal stage stay longer in “Off” state as time elapses even though surgical therapy is attempted. In case where medication to such patients is delayed due to sleep or for other reasons, next dosage administration happens to be difficult due to the mouth and tongue stiffening. Dopamine drug in a liquid state is developed in an attempt to bring the patient back to “on” state, but the way to administer the drug may cause the patient aspiration pneumonia. In such situations there is a need for a tool or method by which we can administer the drug safely, effectively and whenever required. We need a therapy tool or method by which the drug is kept in the body and administered as required.

Blood-Brain Barrier poses a limitation to the administration of helpful therapeutic drugs in treatments of brain tumor, brain infection, and brain granulomatosis. Helpful drugs mostly administered through venous system and mouth reach in cranial cavity through first pass effect and body circulation process which may have ill effect on whole body, but still has yet to pass through the final gateway, Blood-Brain Barrier. For example, Bleomycin is a helpful drug for brain tumor but cannot pass through Blood-Brain Barrier. Accordingly, it is advantageous to administer the drug in other ways than in intravenous or oral medication. Typically a conduit connected with the lesion inside cranial cavity and extended out of brain is used for the drug administration. Length of time for such administration is limited because there is a high risk of infection into the cranial cavity. In this method, the placement of the conduit is allowed for only about a week. A medication tool by which to go over the barrier at the lesion inside cranial cavity and to inject the drug for a long time with little possibility of inflammation is required

International application publications WO/2011/097296 and WO/2011/076382 show the phenomenon of self-sealing closure of a punctured prosthesis due to a member with elastic force and restoration force. International application publication WO/2011/076382 is about a shunt system for hydrocephalus treatment, which has additional function to administer therapeutic drug to the brain lesion using the principle of an Ommaya reservoir. The patent with a drug administration function added to one-direction drainage function which a typical hydrocephalus shunt system shows, has one chamber installed with two offtakes disposed both directions.

U.S. Pat. No. 5,836,935 is about a device which administers drug into cranial cavity or abdominal cavity. In the patent, an installation of a single chamber which can be fully filled again and a guide tube which is inserted into the core of the lesion is disclosed. Mechanism of discharging drug into the lesion is not by change of the chamber's volume but by a passive mechanism related to density of the drug in the chamber. Passive transport which needs a permeable membrane regulating secretion speed and where secretion is made by diffusion or osmotic pressure is different from active transport which the present invention shows and requires energy. The mechanism of drug discharge shown in International application publications WO/2003/070281 and WO/2004/089375 are passive transport.

### Technical Problem

Accordingly, the present invention began with a study on improvement of sexual function of spinal cord injury patients. The present invention provides cure of impotence patients including spinal cord injury patients or improvement of sexual functioning. The present invention also provides one means of treatment of some human and domestic animals' diseases by use of this prosthesis able to intentionally and intermittently secrete stored drug or having control function.

The present invention also provides a prosthesis having a control function of storing drug under human or domestic animals' skin and intentionally and intermittently secreting the drug every time as needed so as to avoid repetitive and invasive medications.

The present invention also provides a prosthesis having a control function as a useful treatment device for impotence of spinal cord injury patients. Advantages can be seen in erection inducing and lasting for spinal cord injury patients without any regulating or interrupting through cord from brain simply by topical application of the present invention, careful spinal reflex and afferent stimulations.

The invention also provides a prosthesis having control function as a means to replace or solve the limitations of corpus cavernosum injection. Side effects affecting penile structure such as fibrosis of penile corpus cavernosum, occurrence of fibroma, and deformation can be avoided and major side effects such as infection, pain and panic can also be avoided. The present invention also provides a means of treatment which can avoid the limitations of a penile prosthesis as a mechanical device.

The present invention also provides a prosthesis having a control function as a means of treatment to avoid the limitations of hypodermic or urethra administration.

The present invention also provides effective solutions to psychological impotence caused by hypersensitivity about penile dwarfism and penile deformation with penile expansion effect which the present invention possesses.

The present invention also provides a prosthesis having a control function as a means to minimize or eliminate such side effects as first pass effect occurring from whole body administration through local administration within the lesions, stimulations on stomach, and dyshepatia and adverse effects on cardio-pulmonary function, motor function, brain nerve function, visual and auditory functions, and psychic function.

The present invention also provides a means that can overcome inconvenience of an invasive administration method that is repeatedly performed every day.

The present invention also provides a prosthesis having a control function, which can overcome a limitation that can occur during a change process from an off-state to an on-state of a Parkinsonian patient

The present invention also provides a prosthesis having a control function, which can achieve a long term placement while avoiding a Blood-Brain Barrier (BBB) by applying the embodiments of the present invention having a drug storage function and a divided administration function in/into the body to a brain disease treatment.

### Technical Solution

In one general aspect, a prosthesis having a control function includes: a base plate implanted under a skin of a human or an animal; a lumen defined by a dome supporting plate formed so as to cover an upper portion and side surfaces of the base plate; at least two domes formed in the dome supporting plate, formed of a flexible material transformable from an inverted U-shape to a U-shape by an external, and having a thin film shape; a chamber formed under the at least two domes; an opening/closing device disposed either in the base plate or in the dome supporting plate and forming a passage through which a drug injected into the chamber and the lumen is secreted according to an application of a pressure to the dome; an injection dome disposed in the dome supporting plate and formed of an elastic body perforated by an injection needle; and a tray disposed under the injection dome.

The opening/closing device may include: an opening/closing hole body having an opening/closing hole at a central portion thereof and inserted into the base plate or the dome supporting plate; a stopper part including a stopper for opening/closing the opening/closing hole and a stopper supporting plate for supporting the stopper; and a grip part connected to the base plate or the dome supporting plate through a connection part at a lower portion thereof, allowing the stopper supporting plate to be inserted into an upper portion thereof, and including a grip outwardly protruding from a side surface thereof to allow a user to insert or take the stopper into/out of the opening/closing hole.

The prosthesis may have an exterior of an I-shape or a C-shape when viewed from top.

The tray may include a sliding preventing member that is uneven.

In another general aspect, a prosthesis having a control function includes: a base plate implanted under a skin of a human or an animal; a lumen defined by a dome supporting plate formed so as to cover an upper portion and side surfaces of the base plate; at least two domes formed in the dome supporting plate, formed of a flexible material transformable from an inverted U-shape to a U-shape by an external, and having a thin film shape; a chamber formed under the at least two domes; an offtake formed by the base plate and the dome supporting plate narrowing and longitudinally extending at one end thereof; an opening/closing device for opening/closing the offtake; an injection dome disposed in the dome supporting plate and formed of an elastic body perforated by an injection needle; and a tray disposed under the injection dome, wherein the opening/closing device forms a passage through which a drug injected into the chamber and the lumen is secreted according to an application of a pressure to the dome.

The opening/closing device may include first and second holding pieces of a plate shape, the holding pieces having a fastening part at a center thereof. The first holding piece may include hooking parts at both ends thereof, and the second holding piece includes hole parts 106 corresponding to the hooking parts. When the first and second holding pieces are coupled to each other and compress the offtake at ordinary times, the both ends thereof may be slidably rotated by a vertical load of a pushing part formed at both end portions of the holding pieces, allowing the first and second holding pieces to be bent in an direction of opening an inlet of the holding piece inlet and thus allowing the offtake to be opened.

The prosthesis may have an exterior of an I-shape when viewed from top.

The tray may include a sliding preventing member that is uneven.

In another general aspect, a bundle of prostheses having a control function include: at least two prostheses including: a base plate implanted under a skin of a human or an animal; a lumen defined by a dome supporting plate formed so as to cover an upper portion and side surfaces of the base plate; at least two domes formed in the dome supporting plate, formed of a flexible material transformable from an inverted U-shape to a U-shape by an external, and having a thin film shape; a chamber formed under the at least two domes; an offtake formed by the base plate and the dome supporting plate narrowing and longitudinally extending at one end thereof; an injection dome disposed in the dome supporting plate and formed of an elastic body perforated by an injection needle; and a tray disposed under the injection dome; a fixing member for fixing the at least two prostheses that are inserted; a collection offtake including a plurality of collection tubes coupled to each of the offtakes formed on the at least two prostheses; a joining part; and an integrated offtake; and an opening/closing device for opening/closing the integrated offtake.

The opening/closing device for the opening/closing the integrated offtake may include first and second holding pieces of a plate shape, the holding pieces having a fastening part at a center thereof. The first holding piece may include hooking parts at both ends thereof, and the second holding piece may include hole parts 106 corresponding to the hooking parts. When the first and second holding pieces are coupled to each other and compress the integrated offtake at ordinary times, the both ends thereof may be slidably rotated by a vertical load of a pushing part formed at both end portions of the holding pieces, allowing the first and second holding pieces to be bent in an direction of opening an inlet of the holding piece inlet and thus allowing the integrated offtake to be opened.

The fixing member may be a matrix including a partition plate for seating a plurality of prostheses vertically formed on a fixing plate and a separation fixing part having a triangular shape to separately fix an upper portion of the prosthesis.

The prosthesis may further include a reinforcing layer disposed in the base plate, the dome supporting plate, and the dome or the injection dome and formed of an elastic material.

The bundle of prostheses may further include a reinforcing layer disposed in the base plate, the dome supporting plate, and the dome or the injection dome and formed of an elastic material.

Other features and aspects will be apparent from the following detailed description, the drawings, and the claims.

### Advantageous Effects

According to research reports by Korean National Rehabilitation Center, about 20% of spinal cord injury patients show satisfaction with sex life and about 75% of the patients need treatments for recovery of sex life. Treatments conducted on the patients include oral medication, urethra medication, hypodermic medication, corpus cavernosum injection, penile prosthesis insertion, vacuum device-induced erection, but the effect of and satisfaction with the treatments are lower for the spinal cord injury patients than for healthy people due to restriction on nervous system from spinal cord damage. In case of a prosthesis equipped with a cylinder and a pump, there can be discrepancy between expected effects from its incurred cost and its actual effect, apart from its probable side effects. The present invention has the effect of resolving the limitations that occur to the patients.

Due to the lost or reduced sense on the part under paralyzed part, infection issue around penis or anus of spinal cord injury patients is ever present. There is high probability that corpus cavernosum injection for them can bring them miserable infection side effect. The side effects of the treatment that they pay for emotional or need satisfaction reduce their adaptability and persistency of the treatment. Application of the present invention has a few more useful effects than surgery for insertion of a prosthesis equipped with a cylinder and a pump or corpus cavernosum injection. The present invention's effect of reducing possibility of infection that can occur to corpus cavernosum injection and reducing side effect of a prosthesis including a machine has great meaning.

The embodiment of the present invention can be supplemental to oral, hypodermic or urethra medication for patients whose levels of satisfaction with the treatments are low in impotence therapy and its own function itself can be effective.

There are areas of therapy other than for impotence where application of the embodiment of the present invention can take effect. With the fewest times of puncture the embodiment can have the effect of as many times of dosages as the number of domes. There are effects of minimizing the limitations of first pass effect, digestive organ or whole body stimulation effect, and delayed medicinal effect.

The embodiment of the present invention can perform its function as an alternative means of administration to frequent skin-damaging hypodermic administration and frequent oral administration because it serves as storage in drug delivery system.

The embodiment of the present invention can function advantageously and effectively to administer growth hormone which requires frequent and long-term injection. Also, it can function effectively to turn Parkinson's disease patients to relaxed “on” state.

The embodiment of the present invention can be effective as an alternative means of treatment to frequent oral medications or injections for pain regulation of patients with uncontrollable and intractable pain from inflammatory fibromyalgia and neuropathic pain and for treatment of osteomyelitis.

The function of the embodiment of the present invention can be effectively used in brain lesions treatment which requires long-term medication in general and includes stereotactic surgery.

### DESCRIPTION OF DRAWINGS

FIG. 1 is a perspective view illustrating a prosthesis having an exterior of I-shape according to a first embodiment of the present invention.

FIG. 2 is a front view illustrating the prosthesis shown in FIG. 1.

FIG. 3 is a cross-sectional view taken along line A-A′ of the prosthesis in FIG. 2.

FIG. 4 is a magnified exploded view three-dimensionally illustrating a portion E of FIGS. 3, 11 and 13.

FIG. 5 is a magnified view illustrating the portion E of FIGS. 3, 11 and 13.

FIG. 6 is a magnified perspective view three-dimensionally illustrating a portion F of FIGS. 3, 11 and 13.

FIG. 7 is a magnified view illustrating the portion F of FIGS. 3, 11 and 13.

FIG. 8 is a perspective view illustrating another exemplary tray in the portion F of FIG. 3.

FIG. 9 is a perspective view illustrating a prosthesis having an exterior of C-shape according to a second embodiment of the present invention.

FIG. 10 is a rear view illustrating a prosthesis shown in FIG. 9.

FIG. 11 is a cross-sectional view taken along line B-B′ of the prosthesis in FIG. 10, where a dome is in an outwardly turned state.

FIG. 12 is a front view illustrating a prosthesis having an exterior of C-shape according to a second embodiment of the present invention, where a dome is in an inwardly turned state.

FIG. 13 is a cross-sectional view taken along line C-C′ of the prosthesis in FIG. 12, where a dome is in an inwardly turned state.

FIG. 14 is a perspective view illustrating a prosthesis having an exterior of I-shape according to a third embodiment of the present invention, where domes are parallelly formed and an opening/closing device is formed in a dome supporting plate, showing a new type of grip part.

FIG. 15 is a front view illustrating the prosthesis shown in FIG. 14.

FIG. 16 is a cross-sectional view taken along line D-D′ of the prosthesis in FIG. 15.

FIG. 17 is a perspective view illustrating a prosthesis having an exterior of I-shape according to a fourth embodiment of the present invention, where an offtake is formed at one end thereof and a holding piece that is an opening/closing device is shown in FIG. 20.

FIG. 18 is a front view illustrating the prosthesis shown in FIG. 17.

FIG. 19 is a cross-sectional view taken along line E-E′ of the prosthesis in FIG. 18.

FIG. 20 is a view illustrating a holding piece serving as an opening/closing device in a prosthesis having an offtake at one end thereof.

FIG. 21 is a perspective view illustrating a bundle of prostheses having an exterior of I-shape according to a fifth embodiment of the present invention, where are connected to each other by matrix and an opening/closing device is shown.

FIG. 22 is a perspective view illustrating a bundle of prostheses having an exterior of I-shape in the prosthesis of FIG. 21.

FIG. 23 is a perspective view illustrating the matrix shown in FIG. 21.

FIG. 24 is a perspective view illustrating an offtake shown in FIG. 21.

FIG. 25 is a cross-sectional perspective view illustrating a reinforcing layer formed in a prosthesis having an exterior of I-shape according to another embodiment of the present invention.

FIG. 26 is a magnified view illustrating a portion G in FIG. 25, where a reinforcing layer is further formed.

### BEST MODE

Hereinafter, exemplary embodiments will be described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience. The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

In some embodiments, prostheses may have an exterior of I-shape. The expression, I-shape, may mean that the prosthesis has a three-dimensional shape but has a long and flat shape resembling the alphabet I.

The expression that the exterior is a C-shape may mean that the prosthesis has a three-dimensional of a ring shape but forms a circular arc, one side of which is opened resembling the alphabet C.

Regarding the shape of a dome, a U-shape and an inverted U-shape may refer to the shape of the dome, corresponding to the shapes of concave lens and convex lens, respectively.

A press-fitting piece may refer to a combination of two structures having a piston shape and a cylinder shape, in which the former is forcibly press-fitted into the latter while having an allowance.

The term, elasticity refers to an extendibility of a degree allowed in embodiments described below and a property of resilience and flexibility of a degree restored to and maintained at the original shape after a paracentesis. Magnetic Resonance Imaging (MRI) compatibility may mean that the prosthesis is not disturbed by photographing due to the unmagnetization of the material of the prosthesis. A highly-frequent invasive administration may mean that drugs having the same properties and effects are injected twice or more a day by an injection method, and a long-term invasive administration may mean that drugs are injected at least for a week or more by the injection method. This numerical limitation may correspond to an estimated value related to the endurance of a patient in terms of administration frequency and administration period. The meaning of the intentional and intermittent drug secretion function may mean a function capable of secreting a desired amount of drug at desired time and place by an intention of a person.

In some embodiments, all portions of the prosthesis may be formed of biocompatible materials. Also, the prosthesis may have MRI compatibility and the X-ray transmission including Computer tomography (CT). The prosthesis may have tolerances to solvent of chemicals, water, vapor, air, acidic materials, and alkalic materials. The prosthesis may have a resistance to the temperature change and a non-permeability to a solution. Furthermore, the prosthesis may comply with regulations about food and human body contact.

Examples of material meeting such designing factors may include fluorinated silicon, fluorinated silicon rubber, and fluorinated silicon plastics. In an embodiment, the prosthesis may be formed of a material selected from silicone, silicone reinforcing agent, silicone rubber, silicone plastics, and polyurethane. SILASTIC and XIAMETER which are patented products of Dow Corning inc. are products that meet the designing factors necessary in embodiments of the present invention, and are being extensively used for plastic prostheses for human body and food containers. The general silicone has limitations in that the mechanical strength is weak, the sealing is released at a high temperature, and the gas permeability somewhat exists. In order to overcome these limitations, fluorinated silicone and fluorinated silicone rubber are used, and these fluorinated products are being used for an opening/closing device of a press-fitting type, a tray of a base part, and an elastic holding piece.

Although some materials have been enumerated in the above description, these are merely examples. Accordingly, other different materials can be used upon manufacturing of the prostheses according to the embodiments if meeting the design factor below.

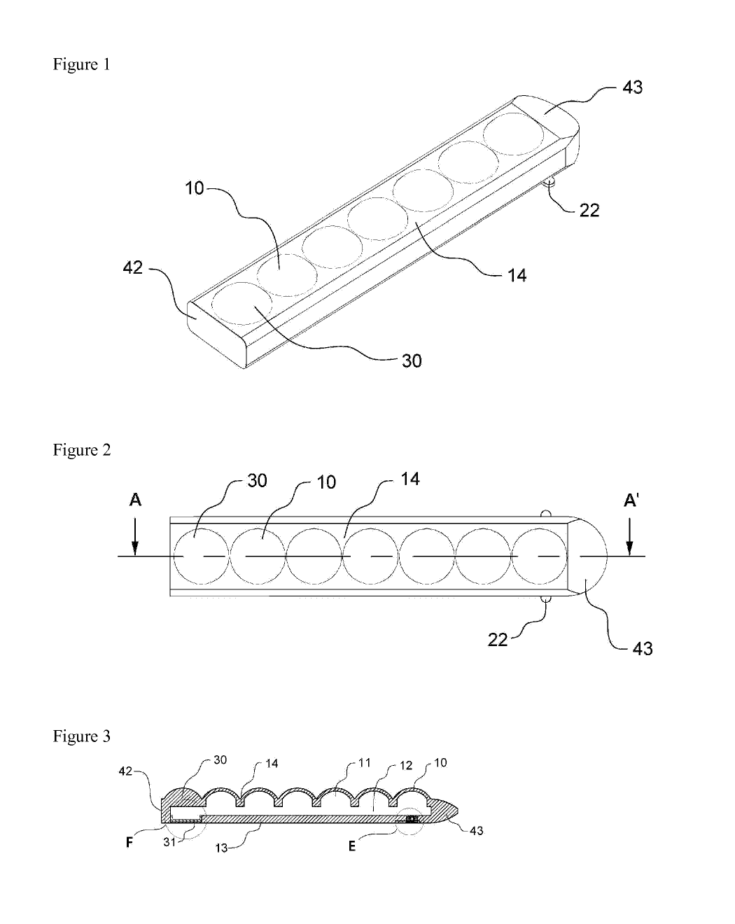

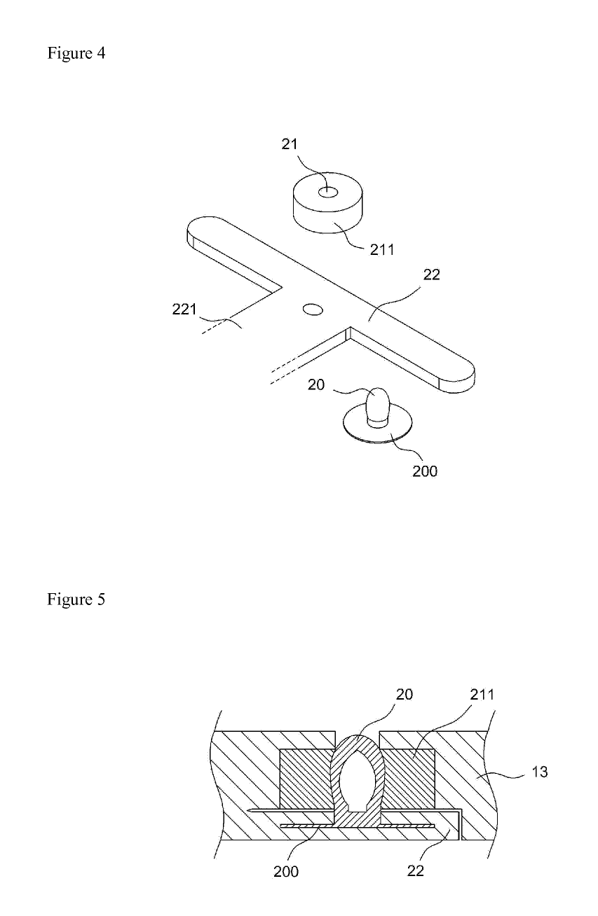

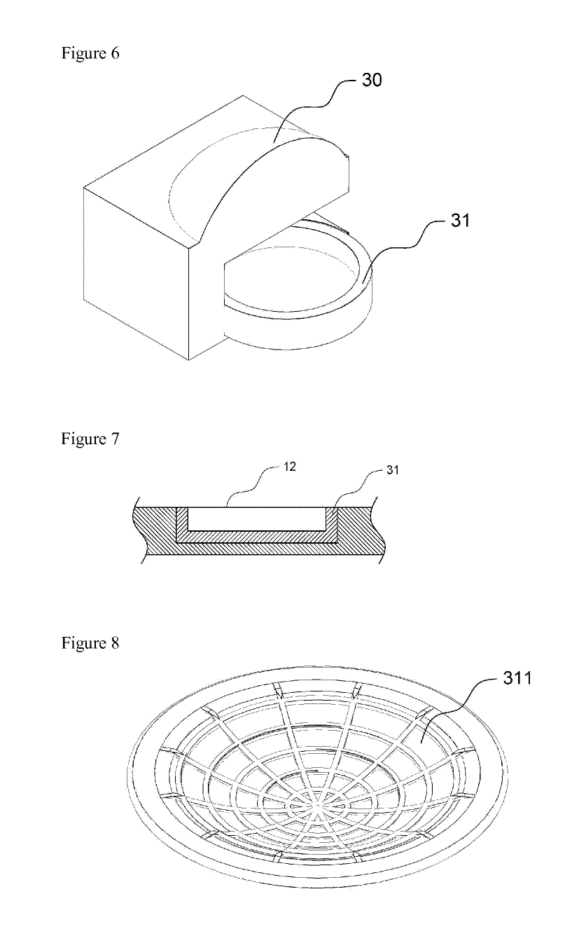

FIGS. 1 to 7 illustrate a prosthesis having an exterior of an I-shape according to a first embodiment. The prosthesis may be implanted under the skin of human or animals. The prosthesis may be implanted under the skin at the proximal of the glans penis and over the tunica albuginea of the corpus cavernosum so as to be parallel to the axis of the penis. The prosthesis may be implanted under the skin of human or animals in addition to the penis. When the prosthesis is implanted under the scalp, the prosthesis may be implanted into the scalp layer. The prosthesis may be implanted under the skin and over the fascia. The prosthesis may be implanted under the skin and over the periosteum. Similarly to the case of penis, the prosthesis may be inserted or implanted under the skin having a thin thickness.

The prosthesis according to the first embodiment may be an elastic body which has a straight-line shape and a length of about 6 centimeters. The prosthesis may have a length of about 1 cm to about 10 cm, about 10 cm to about 15 cm, or about 15 cm to about 30 cm. The prosthesis may have a cross-section of a flat oval shape having a rounded edge, or a cross-section of a circular or rectangular shape. According to the embodiment shown in FIG. 1, the three-dimensional size of the prosthesis may be as follows. The plane length of the prosthesis may be about 60 mm, and the width of the base plate 13 may be about 12 mm. Also, the height from the bottom of the base plate 13 to the top of a dome 10 may be about 8 mm. The thickness of the dome 10 may be about 0.7 mm, and the diameter of the circle formed by a connection between the dome 10 and a dome supporting plate 14 may be about 8.37 mm. Also, the thickness of the dome supporting plate 14 and the base plate 13 may be about 1.75 mm.

FIGS. 3, 11, and 19 illustrate the prosthesis in which a drug is stored before secreted through an opening/closing device, including a chamber 11 and a lumen 12 under the dome 10. A space formed by the chamber 11 and the lumen 12 may be a closed space formed by a connection of the dome 10, the dome supporting plate 14, the base plate 13, an injection dome 30, a tray 31 of an injection device, and an opening/closing device. The two spaces of the chamber 11 and the lumen 12 may be connected to each other, and may not be a space divided by a certain structure. When the dome by the outwardly turned curvature is changed into an inwardly turned curvature form, the capacity of the chamber 11 and the lumen 12 under the dome 10 may be reduced. The change from the dome 10 of an inverted U-shape to the dome 10 of a U-shape may be performed by a pressure intentionally applied to the skin. The pressing force may be usually applied to the dome 10 by the hand.

Two or more domes 10 may be formed in protrusion shape on the dome supporting plate 14 of a planar shape. The dome 10 may be formed in plurality to acquire an effect of drug administration as many as the number of the domes 10 that are formed, just by the skin invasion and pain occurrence of at least one or two times. The dome 10 that is a membrane type and formed of an elastic material may be formed in the intervals of the dome supporting plate in a form corresponding to the curvature of a circular arc attainable by the dome 10. One dome 10 and another dome 10 adjacent thereto may be connected by the dome supporting plate 14. The dome 10 may be a membrane type having a thickness smaller than the dome supporting plate 14, and the chamber 11 and the lumen 12 may be continuously formed under the dome 10. The dome 10 may have a thinner thickness. The dome 10 having a thinner thickness may facilitate the change between the inwardly-turned form and the outwardly-turned form. The thinner thickness may refer to a thin film type in terms of relative criterion compared to the thickness of the dome supporting plate 14 and the base plate 13. In this embodiment, the thickness of the dome 10 may be about 0.7 mm. In other embodiments, the dome may have a thickness of about 0.1 mm. Due to the flexibility and elasticity of the dome 10 of a film type, the dome 10 may be inwardly turned to the chamber 11 and the lumen 12 by an external force applied to the skin. The space of the chamber 11 and the lumen 12 may be reduced by the inward turning of the dome 10, and the amount of drug corresponding to the reduced space may be discharged into the skin. The ideal and preferred shape of the dome 10 may be a spherical shape corresponding to the curvature, but may partially include a straight line. In another embodiment, the dome 10 having a spherical shape may partially include a plane. For example, the top of the dome 10 may be formed in a planar shape. The domes 10 may have the same size as each other. In other embodiments, however, one dome 10 and another dome 10 adjacent thereto may have different sizes. The amount of secretion may be controlled by such changes of the size and shape. The ideal and preferred number of domes 10 may be equal to or greater than 5 and smaller than 30. The number and size of the domes 10 may be determined by the capacity variation of the chamber 11 and the lumen 12, the concentration of a drug filled in the chamber 11 and the lumen 12, the titer of a drug, one-time secretion amount of a drug, the storable period of a drug, and the size of the prosthesis. The domes 10 may be arranged in one straight row on the dome supporting plate 14. However, as shown in FIG. 14, the domes 10 may be arranged in a straight parallel form. The arrangement interval of the domes 10 may be uniform or non-uniform.

The base plate 13 of the prosthesis may be disposed at the opposite side to the side where the dome 10 is disposed when three-dimensionally viewed from the outside. A well-known opening/closing device such as a press-fitting piece may be formed at one portion of the base plate 13. The tray 31 constituting the injection device may be disposed at the edged front end 42 of the base plate 13 at the opposite side. The opening/closing device and the injection device may be disposed around the front end at the opposite side so as to face each other, but may be changed in location within the spirit and scope of the present invention.

The base plate 13 and the dome supporting plate 14 may have the same thickness as each other, and the total thickness of the base plate 13 and the dome supporting plate 14 may be greater than the thickness of the dome 10. The strength of a material forming the base plate 13 and the dome supporting plate 14 may be greater than the strength of a material forming the dome 10. The dome supporting plate 14 may have a strength similar to the base plate 13, and two or more domes 10 may be formed on the flat plane formed by the dome supporting plate 14. The dome supporting plate 14 may resist an external pressure applied to the dome 10 so as to allow only the dome 10 to be turned in position into the chamber 11, and may maintain the whole three-dimensional shape of the prosthesis. The base plate 13 serving as a base and a skeleton of the prosthesis may form the lumen 12 by combining with the dome supporting plate 14. The base plate 13 may be connected to the dome supporting plate 14 at one end thereof, and the one end of the base plate 13 may form a streamlined front end 43 that is gradually reduced in width and thickness. The streamlined front end 43 formed at one end may have a narrowing and piercing shape to help an operator in the insertion or implantation operation.

A drug may be injected into the chamber 11 and the lumen 12 through the injection dome 30. The drug may have a fluidal type such as solution, cream, ointment, microemulsion gel, typical gel, partial liquid, and partial solid, and may be injected by syringe and injection needles. In a case of injection needle of 29 gauges, the outer diameter of the needle cannula may be about 0.3 mm. A needle having a smaller outer diameter may penetrate the injection dome 30 unless the flowability of a drug is restricted in the injection needle. The injection dome 30 may be configured to allow an elastic material to be filled in the chamber 11 under a single dome. The injection dome 30 may form a thick paracentesis portion resistant to an injection needle. The injection dome may be formed of a material that can be penetrated. The elasticity and the restoring force may block a leakage of a drug even though a needle is removed. For example, silicone, silicone reinforced substance, and silicon rubber may prevent a leakage due to elasticity and restoring force thereof. The injection dome 30 may have a sufficient thickness to protect and maintain the leakage prevention function from repetitive paracentesis. The injection dome 30 may take the shape of the dome 10 adjacent thereto, but may be modified in consideration of the location around the front end. The reason why the injection dome 30 exists under the skin upon injection of a drug by an injection needle may become a matter of perception. When the location of the injection dome 30 is changed, the type of the dome 10 may be modified due to the matter of perception. The injection dome 30 and the tray 31 may be disposed so as to form a pair. The injection device including the injection dome 30 and the tray 31 may be formed around the front end to minimize the amount of drug stored in the chamber 11 and the lumen 12 until the drug is secreted. The lumen 12 may be formed under the injection dome 30, and the tray 31 may be embedded into the base plate 13 under the lumen 12.

For example, the tray 31 may be formed of a material such as reinforced silicone plastics, and may block a perforation by an injection needle due to the strength of the tray 31. The tray 31 may have a strength resistant to a perforation by an injection needle. The central portion of the tray 31 facing the lumen 12 may have a concave funnel-shape or U-shape to prevent a slip of the injection needle. As shown in FIG. 8, a tray 311 having a sliding preventing member in which a lattice pattern is formed in an unevenness shape in a rigid body of a disc shape may also be provided. As the sliding preventing member, an uneven structure of a cross pattern or a lattice pattern may be formed in a contact surface with the injection needle. The shape or structure of the tray 31 may be modified within the scope of the present invention. The member of the tray 31 may have a sufficient strength so as not to be penetrated, perforated, cut, and damaged when contacting an acute portion of instrument such as needles, scalpels, and cutting tools. The material constituting the tray 31 may be silicone or silicone plastics, but may be a metal such as carbon steel, aluminum, and titanium. The materials may be materials that are not interrupted from a diagnosis using MRI or X-ray.

The opening/closing device may be a part that allows the amount of drug corresponding to the reduced capacity change of the chamber 11 and the lumen 12 to be secreted under the skin. The opening and closing functions of the opening/closing device for the storage and secretion of the drug may be performed by press-fitting. The opening/closing device may include a stopper 20, an opening/closing hole, and a grip 22. The stopper 20 fitted into the opening/closing hole 21 may block the flow passage, and may allow drug stored in the chamber 11 and the lumen 12 not to leak. The opening/closing hole 21 may be formed in an opening/closing hole body 211, which is embedded into the base plate 13 except the opening/closing hole 21. The stopper 20 may be formed in a stopper supporting plate 200, which is embedded into the grip 22 or 222 except the stopper 20. The grip 22 may be continuously connected to the base plate 13 through the base plate 13 and a grip connection part 221. This connection may be achieved by fusion. In this embodiment, the stopper 20 may have elasticity and flexibility, and the opening/closing hole 21 may maintain stiffness.

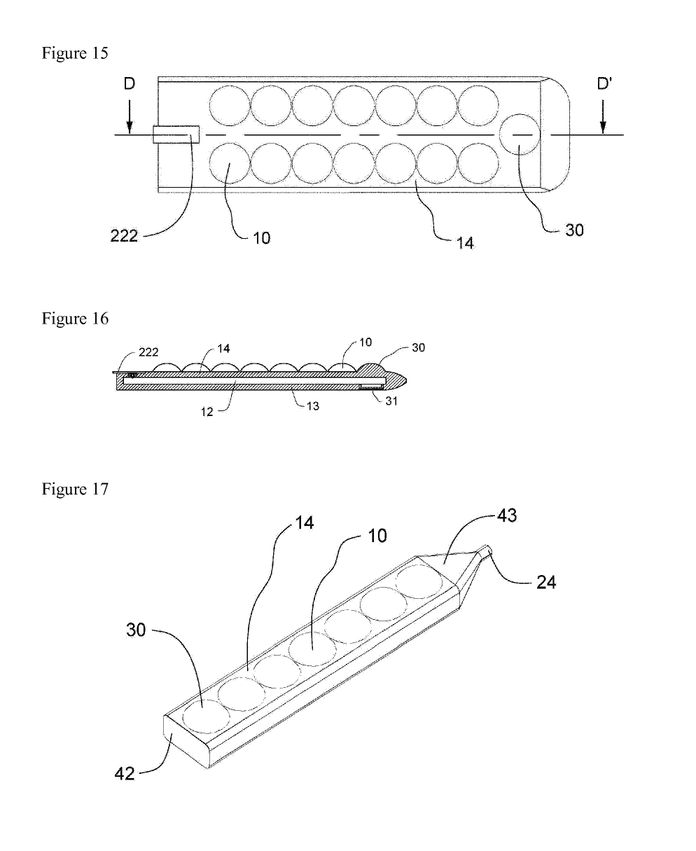

Meanwhile, the opening/closing device may be formed in the dome supporting plate 14 as well as in the base plate 13. In the third embodiment shown in FIGS. 14 to 16, the grip 222 of the opening/closing device may be formed in the dome supporting plate 14.

The opening/closing device may be formed of a material such as reinforced silicone plastics having elasticity. The material constituting the press-fitting piece may have a plastic strength showing elasticity. While the opening/closing device is being intentionally opened, the dome 10 may be inwardly turned into a concave shape. When the opening/closing device is intentionally closed, the drug may be injected through the injection dome 30, and the dome 10 may be outwardly turned into a convex shape due to an increased internal pressure. The opening/closing device may be modified within the spirit and intention of the present invention which discloses a drug is intentionally and intermittently discharged after storage.

In the prosthesis having an I-shape according to the first, third, fourth and fifth embodiments, the opening/closing device may be located at the front end opposite to the injection dome 30. In the prosthesis having a C-shape, the opening/closing device may be located at the front end opposite to the injection dome 30, but may be located at the center of both injection domes 30 as shown in the second embodiment. This is to minimize the amount of drug stored in the chamber 11 and the lumen 12 until the drug is secreted.

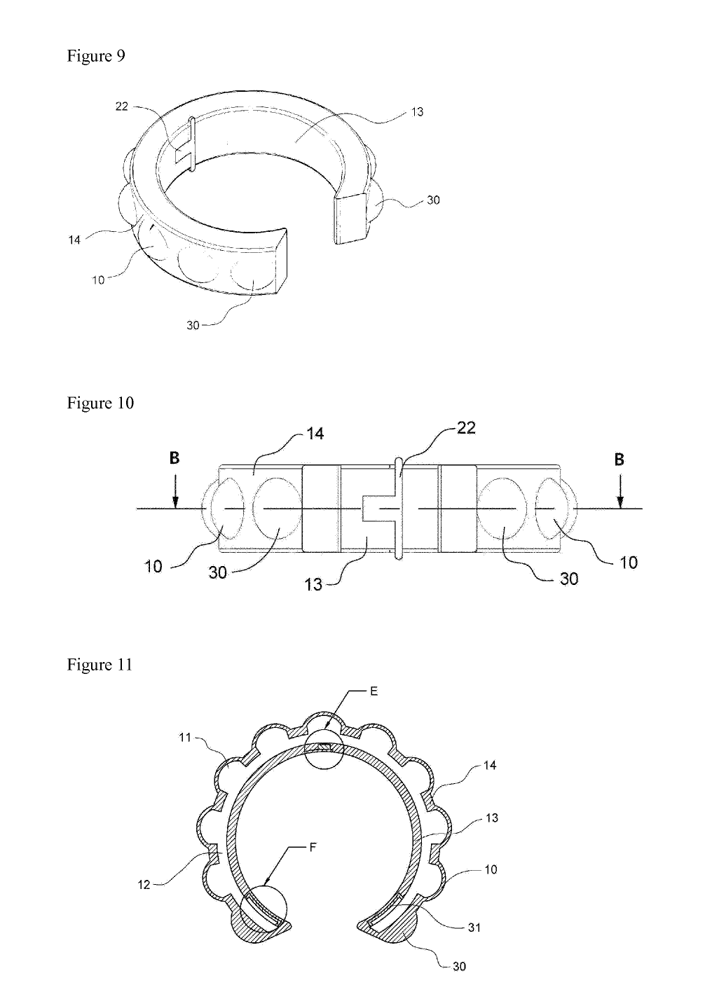

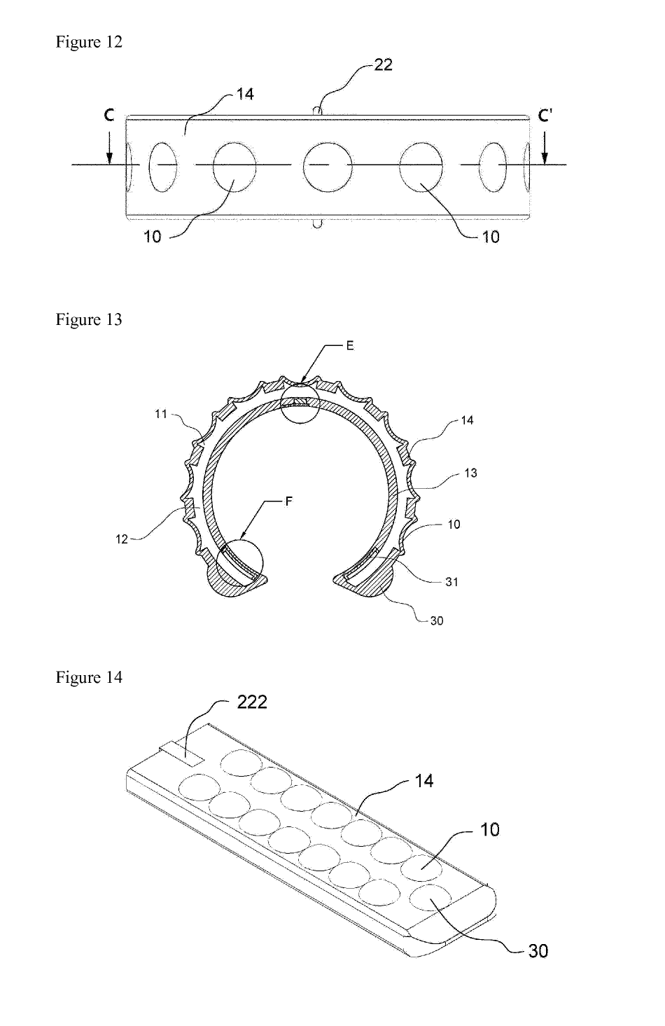

FIGS. 9 to 13 illustrate a prosthesis having an exterior of a C-shape according to the second embodiment.

This prosthesis may have a ring shape with one side opened, in which the prosthesis having an I-shape is rolled to the base plate.

The base plate 13 of the prosthesis may correspond to an internal arc of the prosthesis, and this portion may be located around the tunica albuginea of the corpus cavernosum in the penis.

A well-known opening/closing device such as a press-fitting piece may be formed around the central portion of the base plate 13. A tray 31 constituting the injection device may be disposed at both ends of the base plate 13. The location of the opening/closing device may be located at the center of the front end, and the tray 31 embedded into the base 13 plate may be located at both ends thereof, but the locations may be modified within the spirit and scope of the present invention.

The prosthesis having a C-shape, which is a flexible elastic body, may have a length of about 10 cm when spread in a straight line. In other embodiments, the prosthesis may have a length of about 1 cm to about 10 cm, about 10 cm to about 15 cm, or about 15 cm to about 20 cm. When the prosthesis is cut while being spread, the prosthesis may have a cross-section of a flat oval shape having a rounded edge, or a cross-section of a circular or rectangular shape. In the second embodiment of FIGS. 9 to 13, when the prosthesis is straightly spread, the length of the prosthesis may be about 100 mm, and the width of the base plate 13 may be about 12 mm. Also, the height from the bottom of the base plate 13 to the top of a dome 10 may be about 8 mm. The thickness of the dome 10 may be about 0.7 mm, and the diameter of the circle formed by a connection between the dome 10 and a dome supporting plate 14 may be about 8.37 mm. Also, the thickness of the dome supporting plate 14 and the base plate 13 may be about 1.75 mm. However, according to a demand of an operator or a subject, other embodiments may be manufactured so as to have different three-dimensional sizes.

FIGS. 14 to 16 illustrate a prosthesis having an exterior of an I-shape according to a third embodiment. Domes 10 may be arranged in parallel, and an opening/closing device may be disposed in a dome supporting plate 14 The opening/closing device disposed in the dome supporting plate 14 may directly contact the skin to further facilitate the opening and closing. A different type of grip 222 may constitute the opening/closing device. The grip 222 may be disposed at the front end of the prosthesis unlike the grip 22 disposed at the side surface of the prosthesis. The grip 22 or 222 gripped by the hand upon opening/closing operation may have a high stiffness. The prosthesis according to this embodiment may suggest that the domes 10 can be formed in two or more rows.

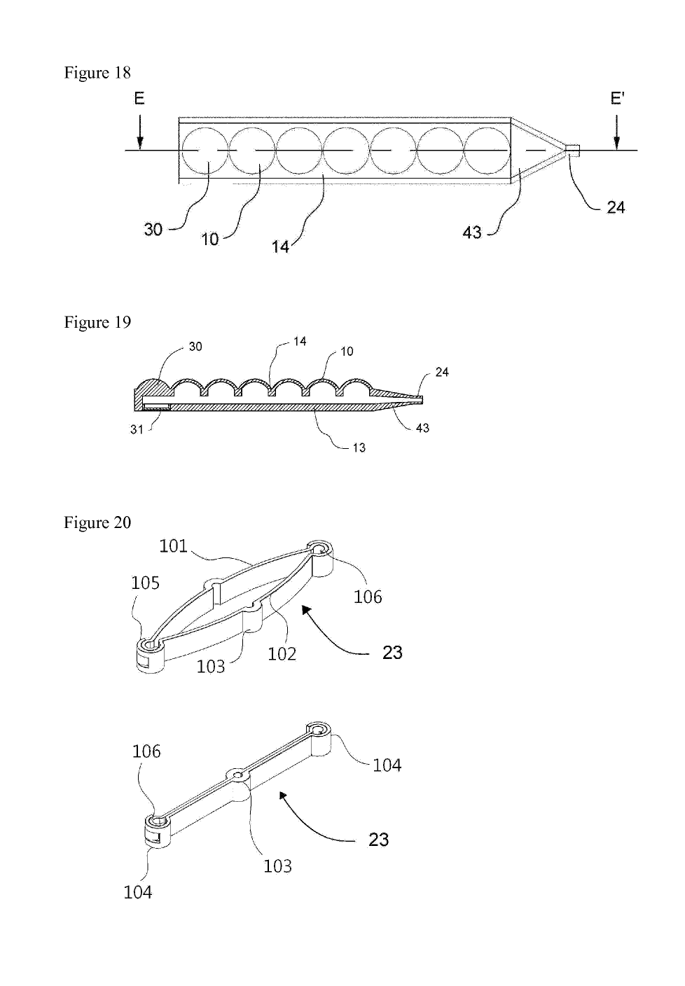

In the fourth embodiment of FIGS. 17 to 19, a prosthesis having an exterior of I-shape according to this embodiment may be similar to the prosthesis having the exterior of an I-shape and a C-shape in terms of structural characteristics such as structure, use method, and material and functional characteristics such as function, effect, and nature, except an offtake 24 or 244 and the opening/closing device for opening/closing the offtake 24 or 244. A detailed description of the structural characteristics and the functional characteristics will be omitted herein, and the offtake 24 or 244 and the opening/closing device will be described in detail.

The offtake 24 may be formed on a streamlined front end 43 at the opposite side of the injection dome 30, and the base plate 13 and the dome supporting plate 14 may be downsized and longitudinally extended. The offtake 244 may be formed on a streamlined front end 43 at the opposite side of the injection dome 30, and the base plate 13 and the dome supporting plate 14 may be downsized, longitudinally extended, and then integrated into one outflow tube.

The tapered and elongated offtake 24 and 244 may become a means for connecting between one structure and another structure. The offtake 24 or 244 may be a structure for enabling a connection with an external conduit (e.g., Ommaya catheter). At a location where the prosthesis is connected to the external conduit, a holding piece 23 shown in FIG. 20 as the proximal of the prosthesis may be used as an opening/closing member. The offtake 24 or 244 may be an elastic body, and may be sealed by a compression.

The holding piece 23 having elasticity and used as a member for opening/closing the offtake 24 or 244 may have a fastening part 103 at a central portion of the offtake 23. The holding piece 23 may include a first holding piece 101 and a second holding piece 102, which have a plate shape. The first holding piece 101 may include hooking parts 105 at both ends thereof, and the second holding piece 101 may include hole parts 106 corresponding to the hooking parts 102. When the two holding pieces 101 and 102 are coupled to each other and compress the offtake 24 or 244 at ordinary times, the both ends may be slidably rotated by a vertical load of a pushing part 104, allowing the two holding pieces 101 and 102 to be bent in an direction of opening the inlet and thus allowing the offtake 24 or 244 to be opened. In this case, the holding piece 23 may be formed of a shape memory elastic body, and may include titanium or silicone reinforced substance. The sealing of the lumen 12 may be achieved by a compression force occurring on an elastic part having a restoring ability to the original state and acting on surface parts that are engaged with each other. The opening of the lumen 12 using the holding piece 23 may be performed by a pressure of the hand applied to the pushing part 104 at the both ends of the holding piece 23. When the lumen 12 is opened, the drug may be discharged by the inward turning of the dome 10.

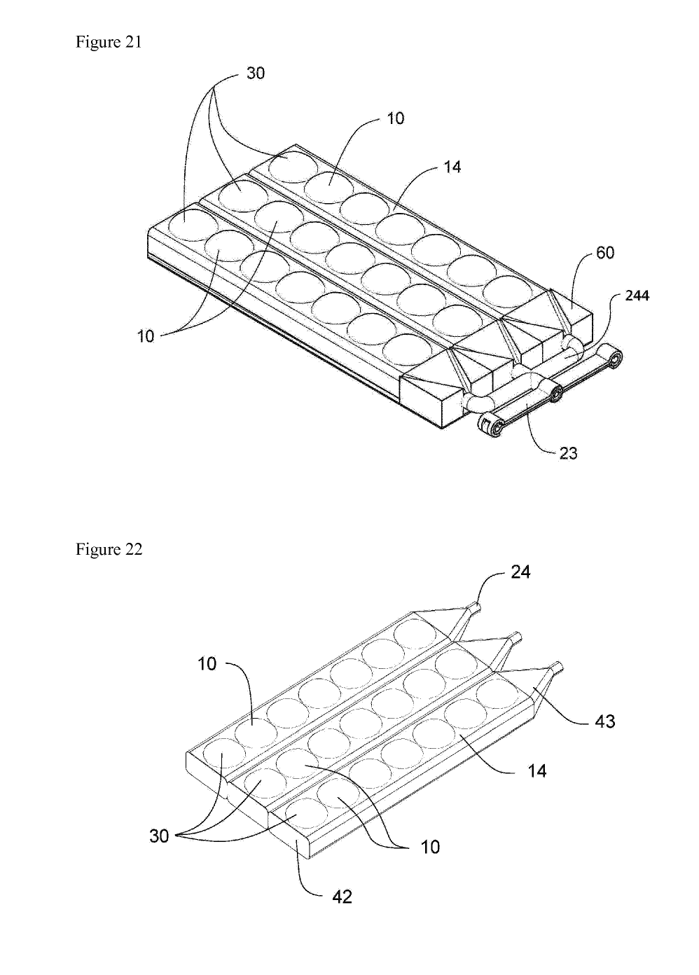

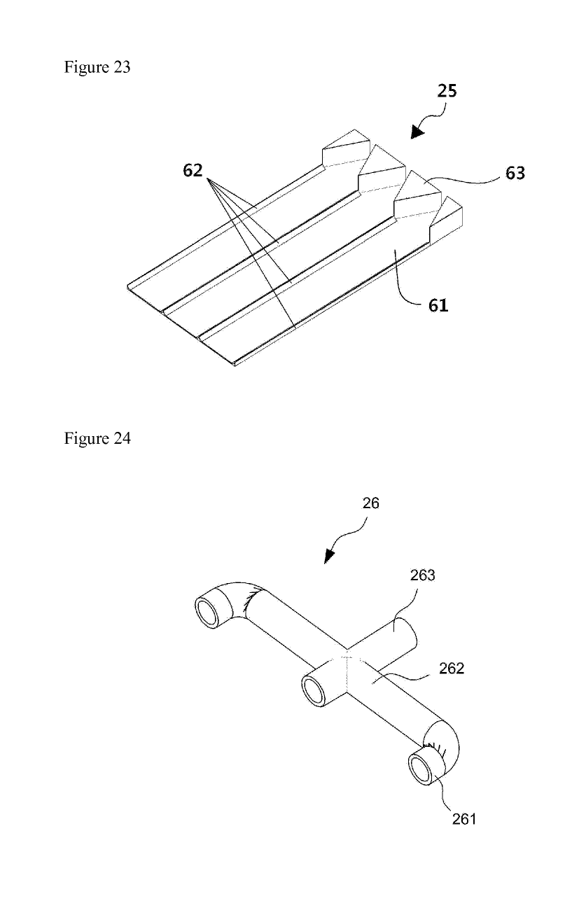

The prosthesis according to embodiments of the present invention may form a bundle of prostheses that include a plurality of prostheses. FIGS. 21 to 24 illustrate a bundle of prostheses according to a fifth embodiment, which show a matrix 25 and a collection offtake 26 as an opening/closing device. In order to form a bundle of prostheses, a fixing member may be provided to fix two or more prostheses that are inserted. The fixing member may include a binding part at a side surface thereof. Also, as shown in FIG. 23, a partition plate 62 may be provided to seat the plurality of prostheses onto a fixing plate 61, and a matrix 60 with a separation fixing part 63 having a triangular shape may be provided to separately fix the upper portion of the prosthesis.

The collection offtake 26 may be needed to collect and discharge drugs discharged from each prosthesis fixed in the bundle of prostheses. The fixing member may use a coupling member formed at a side surface of the prosthesis, and may use the matrix 25 shown in FIG. 23. The matrix 25 may include a partition plate to seat the plurality of prostheses onto a fixing plate, and a separation fixing part having a triangular shape to separately fix the upper portion of the prosthesis. Thus, the plurality of prostheses can be fixed. The matrix 60 may be a thin film type, and may be formed of the same material as a material forming the prosthesis. The matrix 60 may be connected so as to cover each prosthesis except the dome 10 and the offtake 244. The collection offtake 26 may include a plurality of collection tubes 261 coupled by press-fitting to the offtakes 24 formed on two or more prostheses, and a holding piece for opening and closing an integrated offtake 263 for discharging. The collection tubes 261 may be integrated at a joining part 262, and may be connected to the integrated offtake 263.

Each prosthesis constituting the bundle of prostheses may store different drugs, which can be together discharged or selective discharged through the integrated offtake 263.

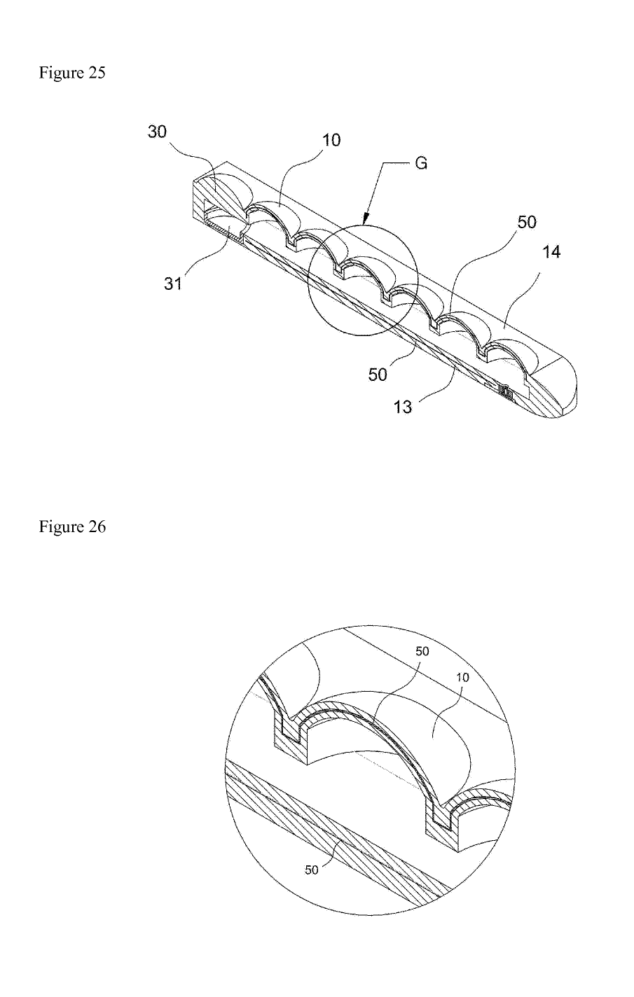

The constitution material of the embodiments of the present invention must meet the design factors proposed in the technical solution. The above-mentioned exemplary materials are considered as sufficiently meeting the conditions in terms of stability, durability, and elasticity, but there are embodiments further reinforced in flexibility, stability, durability, and elasticity through a reinforcing layer added to the materials. There is proposed an embodiment further including a reinforcing layer 50 in each type of prosthesis. FIGS. 25 and 26 are cross-sectional perspective views illustrating a reinforcing layer 50 formed in a prosthesis having an exterior of I-shape according to another embodiment of the present invention. FIG. 26 is a magnified view illustrating a portion G in FIG. 25, where the reinforcing layer 50 is further formed.

For example, a prosthesis may further include the reinforcing layer 50 by forming a silicone layer as the first layer, a fabric reinforced layer as the second layer, and a silicone layer thereon. The reinforcing layer 50 may include one of a mesh plate, a fabric plate, a thin plate, and a film plate based on fineness, and may be selectively embedded into the dome 10, the dome supporting plate 14, and the base plate 13. The reinforcing layer 50 may be dividedly or continuously included in all or a portion of prosthesis according to an embodiment of the present invention.

Like a typical paper, a thin reinforcing layer may include at least one of polyethylene, polypropylene, polyurethane, polyamide (nylon), polycarbonate, other appropriate materials, or a combination thereof. The reinforcing layer 50 may have sufficient strength, flexibility and elasticity for an injection needle to pass therethrough. For example, the reinforcing layer 50 may include spandex that is a highly elastic urethane fiber. The reinforcing layer 50 may serve to maintain the stability and shape of the prosthesis by resisting an external force applied to the prosthesis, particularly, to the dome 10.

The base plate 13 and the dome supporting plate 14 may be manufactured by stages or may be manufactured integrally with each other. Particularly, the stopper supporting plate 200 and the opening/closing hole body 211 may be embedded into the base plate 13 or the dome supporting plate 14, or may be manufactured integrally with the base plate 13 or the dome supporting plate 14. The formation process of the injection dome 30, the connection formation process of the dome 10 and the dome supporting plate 14, the connection formation process of the injection dome 30 and the dome supporting plate 14, and the connection formation process of the tray 31 and the base plate 13 may be similar to the above-mentioned process.

The manufacturing or the manufacturing steps may be achieved using appropriate means well-known in the art.

Hereinafter, a method for using a prosthesis having a control function according to an embodiment present invention will be described.

First, a prosthesis may be prepared, and then may be implanted under the skin.

In this case, the opening/closing device of the prosthesis may be opened, and the shape of the dome 10 of the prosthesis may be changed into a U-shape.

Thereafter, the opening/closing device of the prosthesis may be closed, and a drug may be injected through the skin and the injection dome 30 of the prosthesis. Thus, the shape of the dome 10 may be changed into an inverted U-shape due to a pressure increase of the chamber 11 and the lumen 12 inside the prosthesis.

The step of opening the opening/closing device of the prosthesis, the step of changing the shape of the dome 10 of the prosthesis into the U-shape, and the step of closing the opening/closing device of the prosthesis may be performed only by a force of the hand.

The step of changing the shape of the dome 10 into the inverted U-shape due to the pressure increase of the chamber 11 and the lumen 12 inside the prosthesis may be achieved by an injection of a drug using a syringe or injection needle. The injection dome 30 may be recognized, and then the drug may be injected through the injection needle penetrating the skin and the injection dome. In this case, a pressure outwardly turning the dome 10 may be applied to the lumen 12 and the chamber 11 through the syringe.

A step of inserting or implanting the prosthesis into the skin may include a surgery performed under anesthesia.

In case where the present invention is applied to the human body, the insertion step will be described as follows.

First, the thin skin may be incised by a length of about 12 mm diagonally to an insertion direction of the prosthesis. An operator may form a tunnel under the skin after separating the skin and tissues under the skin through the incised part using mosquito forceps. The prosthesis according the embodiment of the present invention may be inserted into an implantation part in which the tunnel is formed using forceps. The incision and tunnel sizes may vary according to the three-dimensional size of the prosthesis.

Meanwhile, the prostheses according to the embodiments of the present invention may be applied to the human body and animals as the following purposes.

The prostheses according to the embodiments can be used for drug administration and treatment in male erectile dysfunction, treatment of hypersensitivity reaction for penis dwarfism and dwarfism, drug administration in conversion from off-state to on-state for the treatment of Parkinson's syndrome, drug administration in treatment necessary for multi-frequency and long-time administration, administration of drugs reduced or lost in pharmacological activity in digestive system, drug administration after stereotactic surgery for brain disease, and drug administration in a treatment method of directly administering drugs on a lesion to avoid delayed effect of drug.

Since the prostheses according to the embodiments are designed to store drugs in the chamber 11 and the lumen 12 and intentionally and intermittently secrete the drugs, the prostheses can be utilized for the purposes described above.

The prostheses may store and secrete sexual function enhancer and/or medicine, dopamine and/or dopamine agonist, growth hormone drug and/or human body hormone drug, cardiovascular and/or vascular medicine, steroid preparation, antibiotics, anti-inflammatory drugs, hair growth solution, antihistaminic agent, nonnarcotic analgesic, narcotic analgesic, antiepileptic agent, antitumor agent, skin disease medicine, and antioxidant.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A prosthesis having a control function, comprising:
a base plate implanted under a skin of a human or an animal;
a lumen defined by a dome supporting plate formed so as to cover an upper portion and side surfaces of the base plate;
at least two domes formed in the dome supporting plate, formed of a flexible material transformable from an inverted U-shape to a U-shape by an external force, and having a thin film;
a chamber formed under the at least two domes;
an opening/closing device disposed either in the base plate or in the dome supporting plate and forming a passage through which a drug injected into the chamber and the lumen is secreted according to an application of a pressure to the dome;
an injection dome disposed in the dome supporting plate and formed of an elastic body configured to be perforated by an injection needle; and
a tray disposed under the injection dome.

2. The prosthesis of claim 1, wherein the opening/closing device comprises:
a body having an opening/closing hole at a central portion thereof and inserted into the base plate or the dome supporting plate;
a stopper part comprising a stopper for opening/closing the opening/closing hole and a stopper supporting plate for supporting the stopper; and
a grip part connected to the base plate or the dome supporting plate through a connection part at a lower portion thereof, allowing the stopper supporting plate to be inserted into an upper portion thereof, and comprising a grip outwardly protruding from a side surface thereof to allow a user to insert or remove the stopper into/out from the opening/closing hole.

3. The prosthesis of claim 2, wherein the prosthesis has an exterior of an I-shape or a C-shape when viewed from top.

4. The prosthesis of claim 3, wherein the tray comprises a sliding preventing member that is uneven.

5. The prosthesis of claim 1, further comprising a reinforcing layer disposed in the base plate, the dome supporting plate, and the dome or the injection dome and formed of an elastic material.

6. A prosthesis having a control function, comprising:
a base plate implanted under a skin of a human or an animal;
a lumen defined by a dome supporting plate formed so as to cover an upper portion and side surfaces of the base plate;
at least two domes formed in the dome supporting plate, formed of a flexible material transformable from an inverted U-shape to a U-shape by an external, and having a thin film;
a chamber formed under the at least two domes;
an offtake formed by the base plate and the dome supporting plate narrowing and longitudinally extending at one end thereof;
an opening/closing device for opening/closing the offtake;
an injection dome disposed in the dome supporting plate and formed of an elastic body configured to be perforated by an injection needle; and
a tray disposed under the injection dome,
wherein the opening/closing device forms a passage through which a drug injected into the chamber and the lumen is secreted according to an application of pressure to the dome.

7. The prosthesis of claim 6, wherein:
the opening/closing device comprises first and second holding pieces of a plate shape, the holding pieces having a fastening part at a center thereof;
the first holding piece comprises hook parts at both ends thereof, and the second holding piece comprises hole parts corresponding to the hook parts; and
when the first and second holding pieces are coupled to each other and compress the offtake at ordinary times, the both ends thereof are slidably rotated by a vertical load of a pushing part formed at both end portions of the holding pieces, allowing the first and second holding pieces to be bent in a direction of opening an inlet of the holding piece inlet and thus allowing the offtake to be opened.

8. The prosthesis of claim 6, wherein the prosthesis has an exterior of an I-shape when viewed from top.

9. The prosthesis of claim 6, wherein the tray comprises a sliding preventing member that is uneven.

10. The prosthesis of claim 6, further comprising a reinforcing layer disposed in the base plate, the dome supporting plate, and the dome or the injection dome and formed of an elastic material.

11. A bundle of prostheses having a control function, comprising:
at least two prostheses comprising: a base plate implanted under a skin of a human or an animal; a lumen defined by a dome supporting plate formed so as to cover an upper portion and side surfaces of the base plate; at least two domes formed in the dome supporting plate, formed of a flexible material transformable from an inverted U-shape to a U-shape by an external force, and having a thin film;
a chamber formed under the at least two domes; an offtake formed by the base plate and the dome supporting plate narrowing and longitudinally extending at one end thereof; an injection dome disposed in the dome supporting plate and formed of an elastic body configured to be perforated by an injection needle;
and a tray disposed under the injection dome;
a fixing member for fixing the at least two prostheses that are inserted;
a collection offtake comprising a plurality of collection tubes coupled to each of the offtakes formed on the at least two prostheses; a joining part; and an integrated offtake; and
an opening/closing device for opening/closing the integrated offtake.

12. The bundle of prostheses of claim 11, wherein:
the opening/closing device for the opening/closing the integrated offtake comprises first and second holding pieces of a plate shape, the holding pieces having a fastening part at a center thereof;
the first holding piece comprises hook parts at both ends thereof, and the second holding piece comprises hole parts corresponding to the hook parts; and
when the first and second holding pieces are coupled to each other and compress the integrated offtake at ordinary times, the both ends thereof are slidably rotated by a vertical load of a pushing part formed at both end portions of the holding pieces, allowing the first and second holding pieces to be bent in a direction of opening an inlet of the holding piece inlet and thus allowing the integrated offtake to be opened.

13. The bundle of prostheses of claim 11, wherein the fixing member is a matrix comprising a partition plate for seating a plurality of prostheses vertically formed on a fixing plate and a separation fixing part having a triangular shape to separately fix an upper portion of the prosthesis.

14. The bundle of prostheses of claim 11, further comprising a reinforcing layer disposed in the base plate, the dome supporting plate, and the dome or the injection dome and formed of an elastic material.
